(19) United States
(12) Patent Application Publication
(10) Pub. No.: US 2025/0177037 A1
(43) Pub. Date: Jun. 5, 2025

(54) MULTI-ELECTRODE ASSEMBLY FOR HYBRID MAPPING AND ABLATION CATHETER

(71) Applicant: St. Jude Medical, Cardiology Division, Inc., St. Paul, MN (US)

(72) Inventors: Greg OLSON, Elk River, MN (US); Timothy MARASS, Minneapolis, MN (US); Andrew OLIVERIUS, Zimmerman, MN (US)

(21) Appl. No.: 18/845,278

(22) PCT Filed: Mar. 28, 2023
(86) PCT No.: PCT/US2023/065038

§ 371 Date: Sep. 9, 2024

Related U.S. Application Data
(60) Provisional application No. 63/324,791, filed on Mar. 29, 2022.

(51) Int. Cl. A61B 18/14; A61B 18/00

(52) U.S. Cl. CPC A61B 18/1492; A61B 2018/00083; A61B 2018/00577; A61B 2018/00773; A61B 2018/1417

(57) ABSTRACT

A multi-electrode assembly of the present disclosure includes a plurality of electrodes; and an electrode support member; wherein a portion of the electrode support member is adapted to selectively ablate a tissue in contact therewith, and at least a portion of the electrodes are configured and arranged on the electrode support member to detect electrophysiological characteristics of the tissue. The electrode support member can be constructed of flexible material and shaped to facilitate contact with certain anatomical structures (e.g., linear, loop, spiral, planar array, or basket shapes). Embodiments described enable selective activation of the electrode support member for ablation and selective activation of electrodes for electrophysiological mapping and/or ablation.

### CROSS-REFERENCE TO RELATED APPLICATIONS

This application claims priority to provisional application Ser. No. 63/324,791, filed Mar. 29, 2022, which is incorporated herein by reference in its entirety.

### BACKGROUND

The embodiments described herein relate generally to catheter devices for therapeutic electrical energy delivery. In particular, multi-electrode ablation catheters that can be used for therapeutic tissue ablation and electrophysiological mapping are described.

Pulsed field ablation (PFA) utilizes a controlled electric field to ablate and scar tissue through a process called irreversible electroporation (IRE). PFA provides for higher tissue specificity than conventional ablation. For ablation to be successful, detailed mapping and navigation of catheters that deliver energy to specific targets are necessary.

### SUMMARY

Accordingly, a first aspect of the present disclosure features a multi-electrode assembly of a mapping and ablation catheter, the assembly comprising: a first plurality of electrodes; and an electrode support member; wherein a portion of the electrode support member is adapted to deliver ablation therapy, and wherein at least a portion of the first plurality of electrodes are configured and arranged on the electrode support member to detect electrophysiological characteristics of a tissue in contact therewith. The first plurality of electrodes can be selected from the group consisting of ring electrodes, tip electrodes, tip-segment electrodes, split ring electrodes, and ring-segment electrodes. The electrode support member may further comprise a plurality of spot electrodes, printed electrodes, conductive traces, or a combination thereof. The electrode support member may further comprise a tip electrode configured to deliver ablation energy to the tissue. The electrode support member can be configured as a planar array including two or more struts, each of the struts lying in a common plane, wherein at least a portion of the plurality of electrodes are configured and arranged on the struts to detect electrophysiological characteristics of the tissue in contact with the planar array. At least one of the first plurality of electrodes includes electrodes formed on an electrically-insulative substrate positioned on a surface of the strut. The first plurality of electrodes can be selected from the group consisting of ring electrodes, split ring electrodes, and ring-segment electrodes. In some cases, the multi-electrode assembly of further comprises a second plurality of electrodes selected from the group consisting of spot electrodes, printed electrodes, and a combination thereof, wherein the second plurality are configured and arranged to detect electrophysiological characteristics of the tissue in contact therewith. At least one electrode of the first plurality can be positioned between two electrodes of the second plurality, optionally the first plurality of electrodes is selected from the group consisting of spot electrodes, printed electrodes, and a combination thereof. The first plurality of electrodes can be uniformly spaced along the struts. A surface of the struts may include exposed metal and be capable of delivering ablation energy between the at least two struts. The planar array may include at least four struts, and each strut may include at least four electrodes. The electrode support member can be configured as a flexible basket, including a plurality of splines, wherein the first plurality of electrodes are configured and arranged on the splines to detect electrophysiological characteristics of a tissue in contact therewith. At least one of the electrodes can comprise a dielectric layer positioned between the electrode and a spline surface. The first plurality of electrodes of the flexible basket can be selected from the group consisting of ring electrodes, split ring electrodes, and ring-segment electrodes. The flexible basket can further include a second plurality of electrodes mounted to a surface of the splines, the second plurality selected from the group consisting of spot electrodes, printed electrodes, and a combination thereof, wherein the second plurality are configured and arranged to detect electrophysiological characteristics of the tissue in contact therewith. At least one electrode of the first plurality can be positioned between two electrodes of the second plurality. The first plurality of electrodes may include at least two sets of electrodes, wherein a first set of electrodes has a larger total surface area than a second set of electrodes, optionally, the first plurality of electrodes are selected from the group consisting of spot electrodes, printed electrodes, and a combination thereof. A surface of the splines can include exposed metal and be capable of delivering ablation energy between at least two splines. The first plurality of electrodes can include at least two sets of electrodes, wherein a first set of electrodes has a larger total surface area than a second set of electrodes. At least the first set of electrodes can be configured to behave as a single large electrode.

The details of one or more examples are set forth in the description below. Other features, objects, and advantages will be apparent from the description and from the claims.

### BRIEF DESCRIPTION OF DRAWINGS

Various embodiments of the present disclosure are explicated by the accompanying drawings, which are not necessarily drawn to scale. Like numerals having different letter suffixes represent different instances of substantially similar components. Reference is made to illustrative embodiments depicted in the figures, in which:

FIG. 1A is an isometric view of a multi-electrode assembly of a catheter including electrodes coupled to an electrode support member, according to one or more embodiments of the present disclosure.

FIG. 1B is an isometric view of a linear multi-electrode assembly of a catheter including ring and segment electrodes coupled to an electrode support member, according to one or more embodiments of the present disclosure.

FIG. 1C is an isometric view of a multi-electrode assembly of a catheter including an electrode support member and flexible and direct print electrodes, according to one or more embodiments of the present disclosure.

FIG. 1D is an isometric view of a multi-electrode assembly of a catheter including a pair of electrode support members mounted with a plurality of ring electrodes, the assembly configured to generate an electric field for ablation therapy and/or electrophysiological mapping, according to one or more embodiments of the present disclosure.

FIG. 1E is an isometric view of a planar multi-electrode assembly of a catheter including an array of electrodes mounted on a flexible framework of electrode support members (i.e., struts), according to one or more embodiments of the present disclosure.

FIG. 1F is a close-up view of the distal tip portion of multi-electrode assembly 500, according to one or more embodiments of the present disclosure.

FIG. 1G is a plan view of a planar multi-electrode assembly of a catheter including an array of electrodes mounted on a flexible framework of electrode support members (i.e., struts) having exposed interelectrode regions, according to one or more embodiments of the present disclosure.

FIG. 1H is an isometric view of a basket-type multi-electrode assembly of a catheter including an array of electrodes mounted on a flexible framework of electrode support members (i.e., splines) having exposed interelectrode regions, according to one or more embodiments of the present disclosure.

FIG. 2A is an isometric view of a linear multi-electrode assembly of a catheter including at least two sets of electrodes (e.g., durable electrodes and flexible/direct print electrodes), according to one or more embodiments of the present disclosure.

FIG. 2B is a plan view of a planar multi-electrode assembly of a catheter including an array having at least two sets of electrodes (e.g., durable electrodes and flexible/direct printed electrodes) coupled to a framework of electrode support members (i.e., struts), according to one or more embodiments of the present disclosure.

FIG. 2C is an isometric view of a basket-type multi-electrode assembly of a catheter including an array having at least two sets of electrodes (e.g., durable electrodes and flexible/direct printed electrodes) mounted on a flexible framework of electrode support members (i.e., splines), according to one or more embodiments of the present disclosure.

### DETAILED DESCRIPTION

Embodiments of the present disclosure describe a multi-electrode assembly of a hybrid mapping and ablation catheter. Typically, the catheter includes a handle having a proximal end, a shaft having a proximal end portion and a distal end portion. One or more parts of the catheter body can be configured to be inserted into a patient's body and an organ/tissue thereof. The multi-electrode assembly is mounted or otherwise attached to the distal end portion of the shaft and shaped to facilitate contact with certain anatomical structures. In some cases, the shaft of the catheter body is part of the multi-electrode assembly (e.g., when the shaft is configured to have an electrode mounted or otherwise attached thereto.

A multi-electrode assembly of the present disclosure includes a plurality of electrodes and an electrode support member configured or adapted to selectively ablate a tissue in contact therewith. Electrodes of the multi-electrode assembly may be used for a variety of diagnostic and therapeutic purposes including cardiac mapping and/or ablation (e.g., RF ablation and/or PFA ablation), for example. At least a portion of the electrodes are configured and arranged on the electrode support member to detect electrophysiological characteristics of the tissue. The multi-electrode assembly can have a linear, loop, spiral, planar array, or basket shape that is deformable to conform to a patient's vasculature.

The electrode support member can be formed of any flexible, superelastic, pseudoelastic, or semi-rigid (e.g., deformable) material capable of facilitating sufficient contact with the patient's tissue for efficient mapping and/or ablation. Suitable materials include, for example, metal alloys, stainless steel (including spring stainless steel sold under the trade name Elgiloy® and Hastelloy®), copper-aluminum-nickel alloys, CoCrNi alloys (e.g., sold under the trade name Phynox), MP35N®, CoCrMo alloys, alloys including zinc, copper, gold, and/or iron, polymers including any of the above materials, shape memory polymers, and/or combinations thereof. Polymer-based electrode support members can have a tube-like configuration. In some cases, the electrode support member is constructed entirely, or in part, of nitinol, referred to herein as a nitinol-based electrode support member. Nitinol is an approximately stoichiometric alloy of nickel and titanium, which may also include minor amounts of other metals to achieve desired properties. The electrode support member can have any length, shape or configuration suitable or necessary for a desired therapy. In some cases, an electrode support member has a rectangular, square, circular, or elliptical cross-section, or a cross-section that varies along the length of the electrode support member.

The electrode assemblies described herein include various arrangements of electrodes. The electrodes may be activated independently from one another (in an “unganged” configuration) or may be activated together as a larger effective electrode (in a “ganged” configuration). For example, activated independent from one another, the electrodes may be used for mapping applications and/or EGM applications. In contrast, multiple electrodes may be activated together to function as a composite electrode for ablation applications and/or near field impedance navigation applications. Further, different subsets of electrode may be selectively activated (relative to one another) to provide improved control over ablation procedures.

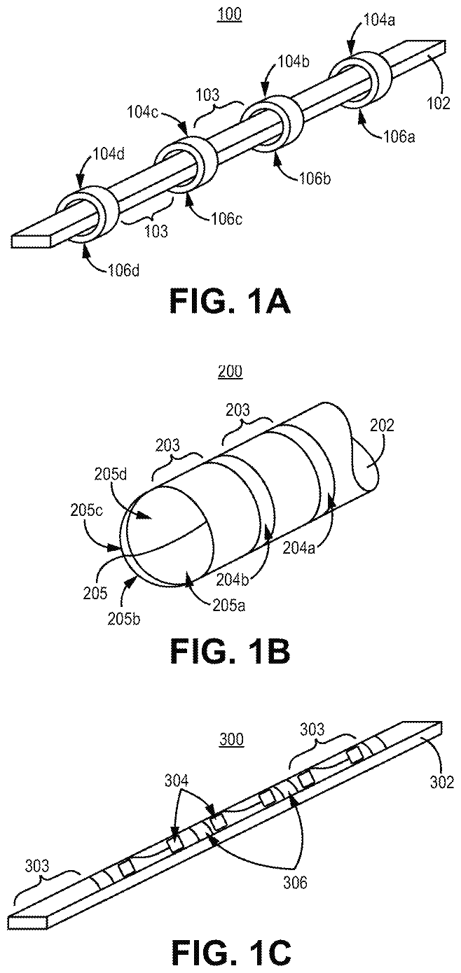

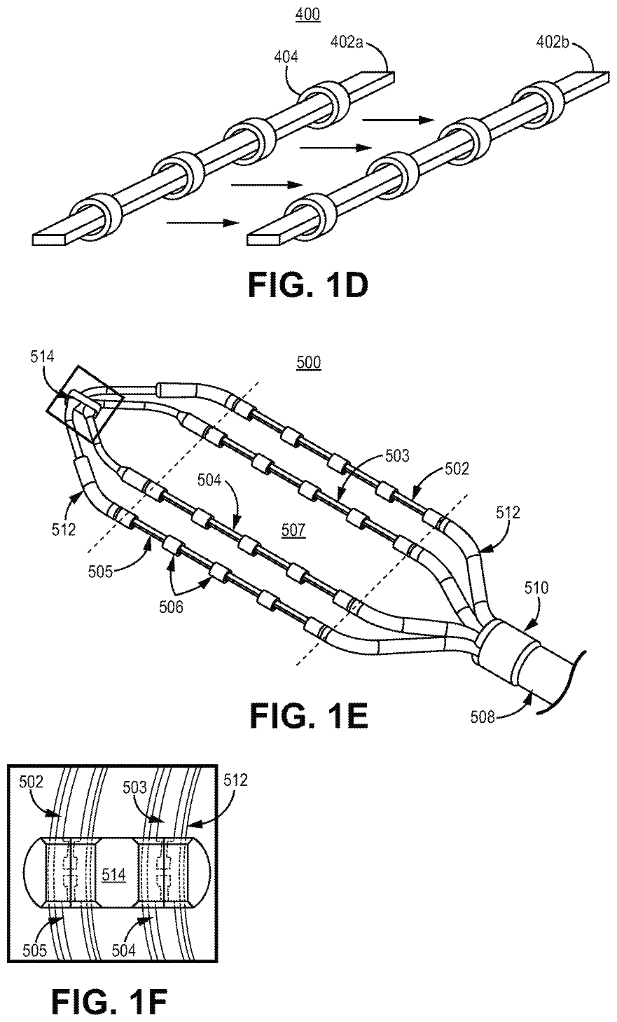

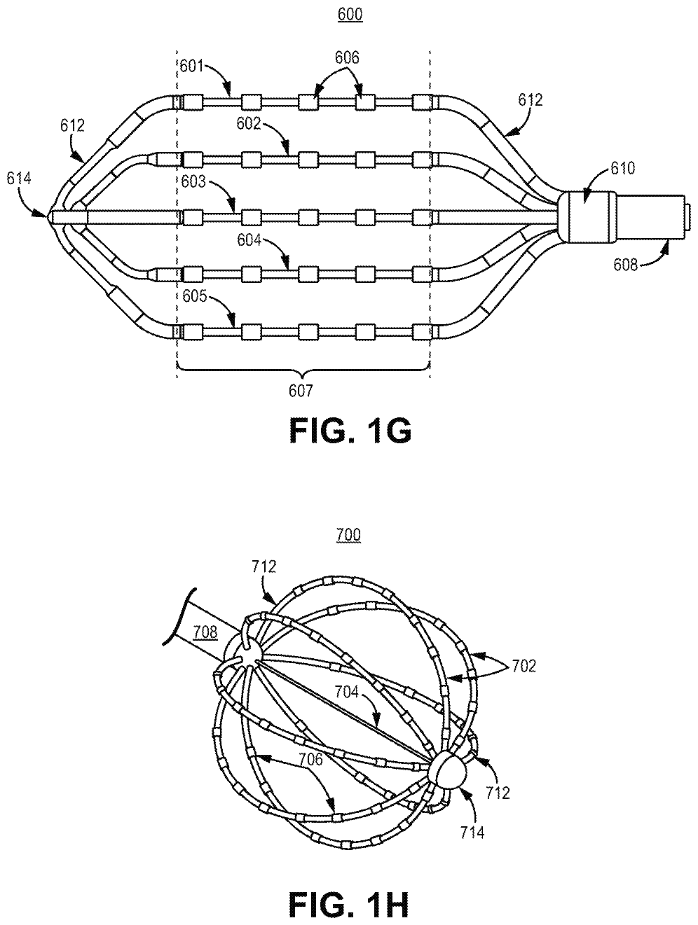

Referring now to FIGS. 1A-1H, a multi-electrode assembly of the present disclosure can be configured to deliver ablation energy to the tissue in contact therewith, via the electrode support member(s).

FIG. 1A shows linear multi-electrode assembly 100 of a hybrid electrophysiology mapping/ablation catheter. Assembly 100 is configured to deliver ablation energy via exposed segments 103 of electrode support member 102, i.e., interelectrode segments that are not covered by an electrically insulating material. Exposed segments 103 are also referred to herein as “therapy ablation members”.

Conductivity of therapy ablation members 103 can be enhanced by the presence of exposed metal on a surface thereof or a metallic core within. In some cases, electrode support member 102 is formed of nitinol, and therapy ablation members 103 further include a core or cladding layer of a conductive alloy such as platinum or gold (not shown). Although the cross-section of electrode support member 102 is depicted as being rectangular, other cross-sectional shapes (e.g., circular, oval, square, or other polygon) are within the scope of the present embodiment. The configuration, shape and size of therapy ablation member 103 can vary along the length of electrode support member 102. The shape, size, and/or configuration of therapy ablation members may impact various parameters of the applied electroporation therapy.

Multi-electrode assembly 100 has mounted thereupon (or otherwise affixed) a plurality of electrodes 104a, 104b, 104c, and 104d along a longitudinal axis configured and arranged on electrode support member 102 to detect electrophysiological characteristics of a tissue. As illustrated, electrodes 104a, 104b, 104c, and 104d are ring electrodes, however one or more of electrodes 104a-d can be a different type of electrode (e.g., split ring electrodes, ring-segment electrodes, or other durable, non-printed electrode). The ring electrodes can be constructed with materials known in the art, and crimped or swaged on to electrode support member 102. Typically ring electrodes, or durable (e.g., non-printed) electrodes described above, can be made of any suitable solid conductive material, such as gold, copper, platinum, iridium, and combinations thereof. Combinations of two or more types of electrode can be mounted on electrode support member 102. A ring electrode with an exposed conductive electrode surface may increase the available surface area of each electrode contacting the patient's tissue. An exemplary exposed electrode surface area can be about 2.5 mm² or less, such as roughly 1 mm²; however, smaller or larger areas can be exposed as dictated by the nature of the catheter and by the electrode's intended application. For example, an electrode can have a small diameter and surface area, such as 0.8 millimeters in diameter with a total surface area of 0.3 to 0.5 mm². Larger surface area electrodes may be used for tissue ablation, while smaller surface area electrodes may be used for mapping and/or localization; however, other configurations are within the scope of this disclosure.

In one or more embodiments, electrodes 104a-d are positioned to enhance electrophysiological activity measurement in the tissue. Thus, the spacing of electrodes may be varied along the longitudinal axis to adjust ablation energy delivery and/or measurement efficiency. For example, as electrodes become smaller and closer together, the electrograms collected thereby will become sharper and more localized evidencing better depiction of local, near-field depolarization of tissue in contact with the electrodes. By varying the size (e.g., width) and spacing of a ring, split ring and ring-segment electrode, different diagnostic and/or therapeutic objectives and/or outcomes can be achieved. Various physical patterns of electrodes 104a-d can be adapted by bonding full ring electrodes to the electrode support member 102 and then covering portions of the electrodes or the support member with an insulating material. Interelectrode spacing can vary along the length of the assembly. The multi-electrode assembly can include any number of electrodes greater than two. For example, the plurality of electrodes can include 3, 5, 8, 12, 72, 84, or more, electrodes (ring, split ring, and/or ring-segment electrodes).

In some cases, electrodes 104a, 104b, 104c, and/or 104d are further configured to deliver ablation energy concurrently with, consecutively to, or independently of, therapy ablation members 103. Thus, both therapy ablation members 103 and one or more of electrodes 104a-d may be individually electrically coupled to an electroporation generator, such as a power supply via suitable electrical wire or other suitable electrical conductors extending through the catheter shaft and may be configured to be selectively energized (e.g., by a power supply and/or computer system) to generate a potential and corresponding electric field between a second electrode support member (not shown) with energized therapy ablation members and/or electrodes.

In some embodiments, for electrophysiology measurements, at least some of electrodes 104a, 104b, 104c, and 104d and therapy ablation members 103 may be activated independent from one another in an “unganged” configuration (i.e., may sense voltages independent from one another, be energized independent from one another, may be energized at different polarities from one another, and/or may be energized at different voltages from one another). In contrast, for ablation applications, two or more of electrodes 104a, 104b, 104c, and 104d and therapy ablation members 103 may be activated in unison in a “ganged’ configuration to form a larger effective electrode. Those of skill in the art will appreciate that any suitable combination of electrodes may be activated in unison.

High-frequency ablation and electroporation generally demand more power and stronger electric fields be delivered to target tissue, which may be accommodated by increasing the effective size of therapy ablation members 103. For example, the size of therapy ablation members 103 can be wider and/or longer as compared to the size of electrodes conventionally used for ablation, sensing, mapping, and diagnostics. In one or more embodiments, multi-electrode assembly 100 is configured (e.g., arranged, spaced, and/or sized) for PFA therapy delivered by therapy ablation members 103, and optionally one or more of electrodes 104a-d. A specific energization pattern of electrodes and ablation members can be implemented to produce a desired lesion surface area, volume, and/or depth, including as areas of continuous or non-continuous lesions, with regularly spaced gaps of untreated tissue.

Electrodes 104a, 104b, 104c, and 104d are formed on (e.g., clad or deposited onto) electrically-insulative substrates 106a, 106b, 106c, and 106d, respectively, and in some embodiments provide electrical isolation between the electrodes 104a, 104b, 104c, and 104d and the electrode support member 102. The electrically-insulative substrates 106a, 106b, 106c can be made of ceramic materials and/or polymers of various durometers. Electrically-insulative substrates 106a, 106b, 106c, and 106d may be the same insulative material, however, one or more of electrically-insulative substrates 106a-d can include a different type of insulative material than the other substrates. Electrically-insulative substrates 106a-d can be formed in various configurations according to the desired use of the catheter. The electrically-insulative substrate can be an injection molded component. For electrodes 104a, 104b, 104c, and 104d, the conductive material can be selectively bonded to, deposited, or coated onto the electrically-insulative substrate by various techniques, including low temperature print manufacturing (with or without sintering), electroplating, electroless plating, sputter deposition, heat, mechanical deformation, cathodic arc deposition, evaporative deposition and pulsed laser deposition or combinations thereof. A particular electrode or segment can independently perform a specialized function (e.g., a mapping function). Thus, each electrode or electrode segment can include the same or different electrode components than an adjacent electrode or electrode segment. In some cases, otherwise independent electrodes and/or segments can also be electrically grouped together to perform an additional function, e.g., to behave as a large electrode for ablation/localization. Thus, in certain embodiments, all of therapy members 103, and electrodes 104a-d may be selectively energized to produce a contiguous pulsed field ablation lesion in a tissue. The lesion produced may be approximately the size of array 100.

In some cases, the electrodes can be electrically coupled with the conductive surface of the electrode support member (i.e., the electrodes do not need to be configured to avoid diversion of current flow to a therapy ablation member). For example, FIG. 1B shows multi-electrode assembly 200 for use on a linear hybrid electrophysiology mapping/ablation catheter, which is configured to deliver ablation energy via therapy ablation members 203 of electrode support member 202. Electrodes 204a and 204b are mounted on the electrode support member without a layer of insulation between the conductive material of the electrode and the conductive material of the support member. Electrodes 204a and 204b are mounted along the longitudinal axis and configured and arranged on the electrode support member 202 to detect electrophysiological characteristics of a patient's tissue. Alternatively, one or more of the electrodes mounted on the electrode support member include a layer insulation, as described in FIG. 1A. Such electrodes can be energized via a separate conductor (e.g., a wire) (not shown).

In other embodiments, electrode 204a and/or 204b is also configured to deliver ablation therapy, thereby increasing the area of the assembly that is capable of delivering ablation energy. A particular electrode can independently perform a specialized function (e.g., a mapping function). Thus, each electrode can include the same or different electrode components than an adjacent electrode or electrode segment. In some cases, otherwise independent electrodes and/or segments can also be electrically grouped together to perform an additional function, e.g., to behave as a large electrode for ablation/localization.

Conductivity of therapy ablation member 203 can be enhanced by the presence of exposed metal on the surface of therapy ablation member 203 as discussed above. One or both of electrodes 204a-b can exhibit more or less effective resistance than therapy ablation members 203 and/or electrode support member 202. As illustrated, electrodes 204a and 204b are ring electrodes, but one or both may be selected from another type of electrode (e.g., split ring electrodes, ring-segment electrodes, or other durable, non-printed electrode). For example, 204a and/or 204b can be segmented to increase the number of electrodes within distal region of the catheter without requiring an increase of the physical area of the region. The number and size of the electrodes can be varied based on the desired therapy (i.e., an electrode support member may include more than two electrodes).

Electrode 204b is positioned proximal to tip electrode 205 at the distal end of assembly 200. Tip electrode 205 can be a single unit or a plurality of discrete electrode segments 205a, 205b, 205c, and 205d. While four segments are illustrated, a tip electrode may include 2 to 10 or more discrete segments. The single unit or segments may be made of the same material and/or similarly durable material as electrodes 204a-b. Tip electrode 205 can be configured for electrophysiological sensing, measurement, diagnostics and/or for therapy delivery. Tip electrode 205 can be constructed from a single conductive material or a multi-layer material of diamagnetic and paramagnetic materials, as described in US2018/0092688A1, the disclosure of which is incorporated herein in its entirety. A particular electrode or segment can independently perform a specialized function (e.g., a mapping function), as discussed above. In some cases, otherwise independent electrodes and/or segments can also be electrically grouped together to perform an additional function, e.g., to behave as a large electrode for ablation/localization.

In some embodiments, for electrophysiology measurements, at least some of electrodes 204a and 204b and electrode segments 205a, 205b, 205c, and 205d may be activated independent from one another in an “unganged” configuration (i.e., may sense voltages independent from one another, be energized independent from one another, may be energized at different polarities from one another, and/or may be energized at different voltages from one another). In contrast, for ablation applications, two or more of electrodes 204a and 204b and electrode segments 205a, 205b, 205c, and 205d may be activated in unison in a “ganged’ configuration to form a larger effective electrode. For example, in one embodiment, electrode segments 205a, 205b, 205c, and 205d are ganged together to form a first effective electrode, and electrodes 204a and 204b are ganged together to form a second effective electrode. Ganging one or more electrodes together may also assist in location determination applications (e.g., in impedance-based location determination systems). Those of skill in the art will appreciate that any suitable combination of electrodes may be activated in unison.

The therapy ablation members may be configured as a bipolar or monopolar electrode assembly for use in bipolar- or monopolar-based electroporation therapy. For example, in FIG. 1B, therapy ablation members 203 may be individually electrically coupled to an electroporation generator, such as a power supply via suitable electrical wire or other suitable electrical conductors extending through the catheter shaft and may be configured to be selectively energized (e.g., by a power supply and/or computer system) with opposite polarities to generate a potential and corresponding electric field therebetween, for PFA therapy. One of therapy ablation members 203 may be configured to function as a cathode, and another one of therapy ablation members 203 may be configured to function as an anode. The configuration, shape and size of the therapy ablation members can vary. The shape, size, and/or configuration of therapy ablation members may impact various parameters of the applied electroporation therapy. In some cases, therapy ablation members 203 are configured to perform unipolar ablation.

For example, multi-electrode assembly 200 may be configured as a monopolar electrode assembly and use a patch electrode (e.g., return electrode, not shown) as a return or indifferent electrode. The current path may be directed from distal tip electrode 205 to a return electrode, in a monopolar therapy application.

Distal tip electrode 205, or a segment thereof (i.e., segments 205a, 205b, 205c, and 205d), can be selectively energized independently of electrodes 204a-b. In some embodiments, distal tip electrode 205 is energized with an opposite polarity to one of therapy ablation members 203, to generate a potential and corresponding electric field therebetween, for PFA therapy. Where distal tip electrode 205 is energized with an opposite polarity to therapy ablation member 203, the electric field generated therebetween may be suitable for point PFA therapy, to precisely target tissue adjacent to distal end of multielectrode assembly 200.

Turning now to FIG. 1C, in one or more embodiments, the plurality of electrodes is selected from flexible and/or direct print electrodes, such as spot electrodes, which can be connected to conductors via flexible circuitry such as printed conductive traces, for example. FIG. 1C shows multi-electrode assembly 300, a hybrid electrophysiology mapping/ablation catheter configured for delivering ablation energy via therapy ablation members 303 of electrode support member 302. As shown, one surface of electrode support member 302 has affixed thereon a plurality of printed conductive traces 304 and a plurality of flexible electrodes 306. Flexible electrodes can wrap around the entire surface of an electrode support member. Portions of electrode support member 302 that do not have an electrode mounted thereupon are identified as therapy ablation members 303, and are configured to deliver ablation energy as described for multi-electrode assemblies 100 and 200. For example, therapy ablation members 303 can be capable of delivering an electric field strength of about 0.1 to 1.0 kV/cm.

As illustrated, electrodes 304 are printed conductive traces and electrodes 306 are flexible and/or direct print electrodes. The alternating arrangement and spacing of conductive traces 304 and flexible electrodes 306 is merely exemplary—other arrangements are within the scope of this embodiment. For example, electrodes 304 and 306 can be positioned to enhance electrophysiological activity measurement in the tissue. As electrodes become smaller and closer together, the electrograms collected thereby will become sharper and more localized evidencing better depiction of local, near-field depolarization of tissue in contact with the electrodes. By varying the size (e.g., width) and spacing of the flexible and/or direct print electrodes, different diagnostic and/or therapeutic objectives and/or outcomes can be achieved. In some cases, a first plurality of flexible electrodes is positioned relative to the electrode support member such that a first set of electrodes is aligned with a first surface of the support member and a second set of electrodes is aligned with a second surface of the support member.

The flexible and/or direct print electrodes 306 can be single sided electrodes or electrodes printed on a flexible, bendable material. Conductive traces 304 can be formed using an ink jet printing process or an additive manufacturing process. In some cases, flexible and/or direct print electrodes 306 are spot electrodes printed on at least one surface of electrode support member 302, using a conductive ink. A similar printing process can also be used to form conductive trace electrodes 304. In one or more embodiments, printed traces 304 can be used to electrically connect flexible electrodes 306 to conductors, or printed traces could function as conductors of multi-electrode assembly 300. Electrical circuit printing techniques to fabricate electrical traces and other circuit components can include automated 3-D printing techniques (including additive and/or removal techniques). Use of flexible and/or printed electrodes permits a higher density of electrodes, allows for complex and complicated physical patterns of electrodes, and does not reduce array flexibility to the extent of non-printed electrodes. Increasing the number of measurement points for electrophysiological activity is advantageous because it substantially reduces the amount of time required to generate an electrophysiology map using an ablation catheter.

A portion of one or both of the pluralities of flexible electrodes 306 or conductive traces 304 is configured for detecting electrophysiological characteristics of the tissue. In some cases, a portion of the conductive traces 304 and flexible electrodes 306 may also be configured as/for use with sensors (e.g., position, pacing, strain, temperature, and the like). The catheter or the multi-electrode assembly can further include components such as, without limitation, a temperature sensor, force sensors, additional sensors or electrodes. For example, a thermocouple can be attached to each electrode to provides temperature readings of the electrode. Flexible and direct printed electrodes for use on a multi-electrode assembly of the present disclosure include those described in US2016/0278923A1, US2017/0112405A1, US2018/0008821A1, US2018/0055631, US2019/0328245A1, US2020/0107878A1, US2021/0022803A1, US2021/0153932A1, WO2018150374A1, and WO2020095250A1, the disclosures of which are incorporated herein in their entireties.

As with the other electrode arrangements described herein, electrodes in multi-electrode assembly 300 may be selectively activated in unganged or ganged configurations to facilitate electrophysiology, ablation, and/or location determination applications as appropriate.

Each of electrodes 306 can be coupled to and communicatively coupled to signal processing circuitry via electrical traces that extend along interior or exterior layers of a flexible printed circuit board (not shown). Flexible and/or direct print electrodes 306 can be disposed upon an electrically insulative layer. In some embodiments, electrode support member 302 may consist of nitinol. In such embodiments, the flexible electrodes 306 may be either bonded directly to the nitinol, or alternatively, directly bonded to polyether block amide (or PEBA) tubing, or tubing of other suitable thermoplastic elastomer tubing which houses the nitinol member internally. Multi-electrode assembly 300 can include any number of flexible electrodes. For example, the plurality of electrodes can include 2, 3, 5, 8, 12, 72, 84 or more flexible and/or direct print electrodes, which may be connected to a sensor or power generator via printed conductive traces.

Referring to FIG. 1D, a plurality of electrode support members can be used as a framework for an array of electrodes. For example, a plurality of electrode support members may be arranged in parallel for use in monopolar (e.g., as part of a system including one or more return electrodes such as patch electrodes) or bipolar-based electroporation therapy. Multi-electrode assembly 400 can be used for hybrid electrophysiology mapping/ablation, and has electrode support members 402a and 402b lying in a common plane, each of which having mounted thereon a plurality of electrodes 404 along a longitudinal axis. Electrode support members 402a and 402b may be individually electrically coupled to an electroporation generator, such as a power supply via suitable electrical wire or other suitable electrical conductors extending through the catheter shaft and may be configured to be selectively energized (e.g., by a power supply and/or computer system) with opposite polarities to generate a potential and corresponding electric field therebetween, illustrated by arrows, for ablation therapy. The configuration, shape and size of the electrode support members can vary, and may impact various parameters of the applied electroporation therapy. Although electrodes 404 are depicted as ring electrodes disposed on a layer of electrically insulative material (see 104a-d and 106a-d in FIG. 1A), one or more of the electrodes can be another type of electrode and/or connector suitable for electrophysiological sensing, measurement, and/or diagnostics (e.g., one or more split ring, ring-segment, spot electrodes, or other printed electrode). The electrodes can be connected via flexible circuits, such as printed conductive traces, as discussed above.

As with the other electrode arrangements described herein, electrodes in multi-electrode assembly 400 may be selectively activated in unganged or ganged configurations to facilitate electrophysiology, ablation, and/or location determination applications as appropriate.

Turning to FIG. 1E, portions of an electrode support member that are not configured for direct delivery of ablation energy can be coated with a dielectric. Multi-electrode assembly 500 includes electrode support members 502, 503, 504, and 505 (referred to as “struts” hereafter) further including a proximal and distal region having outer layer 512 formed of or constructed from an electrically insulating material (e.g., dielectric). Energy delivery region 507 extends between the dashed lines, configured as a 4×5 array of electrodes 506, however other configurations (e.g., 3×8, 5×5, etc.) are within the scope of this embodiment. Suitable material for outer layer 512 includes, without limitation, polyethylene terephthalate (PET), parylene, polyimide (e.g., PI-2771 or HD-4004 available from HD Microsystems), polyether block amide (PEBA) and/or an adhesive (e.g., SU8 epoxy available from MicroChem Corp). Where the electrode support member is made of electrically conductive material, the dielectric can electrically insulate electrodes that are mounted thereon (not shown) from the electrically conductive material. A first region of outer layer 512 extends continuously over a distal portion of struts 502 and 505 and struts 503 and 504 to form a flexible tip joining the respective struts. A second region of outer layer 512 coating each of struts 502-505 extends distally from the coupler to the proximal terminus of energy delivery region 507.

Multi-electrode assembly 500 includes a planar array of electrodes 506 on struts 502, 503, 504, and 505 within energy delivery region 507. Each of struts 502, 503, 504, and 505 carry a plurality of electrodes 506 with the spacing of the electrodes along a length of the strut being the same (or at least known). Other embodiments may include unevenly distributed electrodes along the struts. For example, only a distal portion of the planar array be in contact with the tissue to be treated, a distribution of electrodes 506 may be weighted toward a distal end of assembly 500 to facilitate mapping in the tissue. The struts extend in a plane that is substantially parallel with a longitudinal axis of catheter shaft 508 and are coupled to one another at distal and proximal ends (e.g., at a distal tip of coupler 514 and bushing 510).

Struts 502-505 include interelectrode regions configured to deliver ablation energy. Each of the struts can be precisely, laterally separated from each other to facilitate exact spacing between the exposed interelectrode regions on adjacent struts 502, 503, 504, and 505 and/or between electrodes 506 on adjacent struts 502, 503, 504, and 505. The result is a plurality of electrode and exposed interelectrode regions forming bipole pairs with known spacings. For example, in some embodiments, the center-to-center spacing of a bipole pair may be between 0.5-4 mm, or less than 0.5 millimeters (e.g., 0.1 mm). Alternatively or additionally, the bipole pair spacing can be based on edge-to-edge spacing. Consideration of edge-to-edge spacing may be desirable where the electrodes 506 and/or exposed interelectrode regions of struts 502, 503, 504, and 505 have different relative sizes (or surface areas).

Multi-electrode assembly 500 is configured such that spacing between struts is based on a desired electrode spacing for a given therapy. While the planar array shows 20 electrodes 506, a hybrid electrophysiology mapping/ablation catheter of the present disclosure array may include more or fewer than 20 electrodes. Each strut may have a different number of electrodes than an adjacent strut. In some embodiments, the exposed interelectrode regions on adjacent struts 502-505 may perform unipolar or bipolar ablation (e.g., via the use of bipole pairs of exposed interelectrode regions). This unipolar or bipolar ablation may create specific lines or patterns of lesions. For example, electrodes 506 can be sized and spaced to ensure that the exposed interelectrode regions of the struts are closely spaced and capable of delivering an electric field strength of about 0.1 to 1.0 kV/cm.

Electrodes 506 may receive electrical signals from the heart, or other tissue, which can be used for electrophysiological studies/mapping. For example, electrodes 506 capture electrophysiological information, such as the direction and speed of cardiac signals, to enable the creation of high-density electro maps of cardiac tissue to support optimal treatment. The sampled electrical characteristics may be processed to remove catheter orientation-based signal effects. In some embodiments, the electrodes 506 may perform a location or position sensing function related to tissue mapping (e.g., determine location and/or orientation of the catheter).

Planar multi-electrode assembly 500 is coupled to a distal end of a catheter shaft 508 at a bushing 510 (also referred to as a connector). The catheter shaft 508 may also define a catheter shaft longitudinal axis. In the present embodiment, each of the struts 502-505 extend substantially parallel to the longitudinal axis. The catheter shaft 508 may be made of a flexible material, permitting the catheter to be threaded through vasculature or other body cavity of a patient.

In one or more embodiments, multi-electrode assembly 500 is adapted to conform to one or more specific tissues (e.g., cardiac tissue) and also for delivery/removal of the catheter. For example, collapsibility of materials such as nitinol and/or other flexible substrate facilitates insertion of the planar array into a delivery sheath or introducer, whether during delivery of the catheter into the body or removal of the catheter from the body at the end of a procedure. Each strut 502, 503, 504, and 505 may independently deflect. The construction of struts 502-505, for example, the length and/or diameter of the struts, and material, may be tailored to achieve desired resiliency, flexibility, foldability, conformability, and stiffness characteristics, along a strut or among the plurality of struts.

While multi-electrode assembly 500 is depicted with ring electrodes 506, embodiments having spot, or other flexible/direct-print electrodes coupled to the struts are readily envisioned. The struts of the planar array may comprise flexible thin films compatible with printed circuit manufacturing techniques and/or have such thin films coupled to structural elements of the strut (e.g., nitinol-or polymer based structural elements). Spot electrodes may be printed onto the struts themselves. Printed electrodes may be electrically coupled to signal processing circuitry and/or driver circuitry via traces extending on or within the one or more thin film layers.

Ring electrodes 506 include the same type of electrode or a variety of electrode types. For example, electrodes with smaller surface area may be used exclusively for electrophysiology mapping, while larger surface area electrodes may be used for mapping, tissue ablation, and/or localization. In some cases, the multi-electrode assembly includes one or more slightly enlarged ring electrodes (e.g., for localization of the flexible array in mapping and navigation systems). Each electrode or electrode segment can include the same or different electrode components mounted therein/on as an adjacent electrode or electrode segment. Thus, a particular electrode can independently perform a specialized function (e.g., a mapping function). In some cases, independent electrodes can also be electrically grouped together to perform an additional function, e.g., to behave as a large electrode for ablation/localization.

Electrodes 506 may be used in various bipole configurations to facilitate measurement of electrical characteristics of tissue in contact with the electrodes. Orthogonally-oriented bipole pair combinations are capable of measuring the unique orientation specific electrical characteristics of the tissue in two orthogonal orientations. A first bipole pair may include a pair of electrodes along the length of struts 502, 503, 504, and 505 facilitating the collection of tissue electrical characteristic data in an orientation substantially parallel with the catheter's longitudinal axis. A second, orthogonal bipole pair may extend laterally across adjacent struts 502 and 503, 503 and 504, 504 and 505, facilitating the collection of tissue electrical characteristic data in an orientation substantially transverse to the catheter's longitudinal axis. To facilitate collecting this electrical data, these bipole electrode pairs may be independently addressable by signal processing circuitry. The signal processing circuitry analyzes the received signals from the two sets of bipole pairs to determine orientation independent electrophysiology information of the tissue in contact with the electrodes.

As discussed above, it is possible to drive ablation current between the interelectrode regions of struts 502-505, and optionally between electrodes 506 on adjacent struts concurrently, if desired. The array is configured for bipolar ablation, or, alternatively to drive ablation current in unipolar mode between one or more interelectrode regions and, for example, a patch electrode located on a patient. Unipolar or bipolar ablation therapy techniques utilizing interelectrode regions, with or without one or more of electrodes 506, may be used to create specific lesion lines or lesion patterns.

As with the other electrode arrangements described herein, electrodes in multi-electrode assembly 500 may be selectively activated in unganged or ganged configurations to facilitate electrophysiology, ablation, and/or location determination applications as appropriate.

As seen in FIG. 1E, the distal tip includes coupler 14 joining the flexible joints connecting struts 502 and 505 and connecting struts 503 and 504. The pairs of struts are not electrically connected (shown in greater detail in FIG. 1F), although other embodiments can include other types of connections. Coupler 514 may be constructed from metal or another radiopaque material to permit fluoroscopic visualization. The coupler 514 may facilitate (semi-) independent planar movement between the struts while ensuring the struts are secure within dielectric tubing 512, that the struts maintain the spaced and planar relationship of the array and the electrodes thereon, and permits the struts to be independently energized. For example, struts 502 and 505 can be energized together to form an outer loop and struts 504 and 503 can be energized together to form an inner loop of opposite polarity in one embodiment, or struts 502 and 504 can be energized as cathodes and struts 503 and 505 can be energized as anodes, such that the adjacent struts have opposite polarity. A specific energization pattern of struts and optionally electrodes mounted thereupon can be implemented to produce a desired lesion surface area (e.g., continuous v. non-continuous lesions, with regularly spaced gaps of untreated tissue), volume, and/or depth. Alternatively, one or more of the joints connecting struts 502 and 505 and connecting struts 503 and 504 can electrically connect the respective struts.

With reference to FIG. 1G, a hybrid electrophysiology mapping/ablation catheter of the present disclosure may comprise a planar array with more than four struts. For example, in FIG. 1G, multi-electrode assembly 600 includes five struts, e.g., 601, 602, 603, 604, and 605. Each strut includes a proximal and distal region having outer layer 612 formed of or constructed from an electrically insulating material as described above and an energy delivery region 607 extending between the dashed lines. Energy delivery region 607 is configured as a 5×5 array of electrodes 606, however other array configurations are within the scope of this embodiment. A first region of outer layer 612 extends continuously over a distal portion of struts 601 and 605 and struts 603 and 604 to form a joint of the respective struts. A second region of outer layer 612 extends distally from the coupler to the proximal terminus of energy delivery region 607, coating each of struts 601-605.

Multi-electrode assembly 600 includes an array of electrodes 606. Each of struts 601, 602, 603, 604, and 605 may carry a plurality of electrodes 606 within energy delivery region 607, with the spacing of the electrodes along a length of the planar array being the same (or at least known). The framework formed by struts 601, 602, 603, 604, and 605 is flexible and extends in a plane that is substantially parallel with a longitudinal axis of catheter shaft 608. Struts 601, 602, 603, 604, and 605 are coupled to one another at distal and proximal ends (e.g., at a distal tip 614 and bushing 610). Struts 601, 602, 603, 604, and 605 include interelectrode regions configured to directly deliver ablation energy. Each of the struts can be precisely, laterally separated from each other to facilitate exact spacing between the exposed interelectrode regions on adjacent struts and/or between electrodes 606 on adjacent struts. The result is a plurality of electrode and exposed interelectrode regions forming bipole pairs with known spacings. For example, in some embodiments the center-to-center spacing of a bipole pair may be between 0.5-4 mm, or less than 0.5 millimeters (e.g., 0.1 mm). Alternatively or additionally, the bipole pair spacing can be based on edge-to-edge spacing. Consideration of edge-to-edge spacing may be desirable where the electrodes 606 and/or exposed interelectrode regions of struts 601-605 have different relative sizes (or surface areas). Electrodes 606 can be sized and spaced to ensure that the exposed interelectrode regions of the struts are closely spaced and capable of delivering an electric field strength of about 0.1 to 1.0 kV/cm.

Multi-electrode assembly 600 includes five struts, with spacing between each respective strut based on a desired exposed interelectrode region spacing and/or desired electrode spacing for a given therapy. Additionally, while the planar array shows 25 electrodes 606, a hybrid electrophysiology mapping/ablation catheter of the present disclosure array may include more or fewer than 25 electrodes. Each strut may have a different number of electrodes than an adjacent strut.

In some embodiments, the exposed interelectrode regions on adjacent struts form bipole pairs 601 and 602, 602 and 603, 603 and 604, 604, and 605. Multi-electrode assembly 600 may perform unipolar or bipolar ablation (e.g., via the use of bipole pairs of exposed interelectrode regions). This unipolar or bipolar ablation may create specific lines or patterns of lesions. In some embodiments, electrodes 606 may receive electrical signals from the heart, or other tissue, which can be used for electrophysiological studies/mapping, as described above.

Multi-electrode assembly 600 is coupled to a distal end of a catheter shaft 608 at a bushing 610 (e.g., a connector). The catheter shaft 608 may also define a catheter shaft longitudinal axis. In the present embodiment, each of the struts 601-605 extend substantially parallel to the longitudinal axis. The catheter shaft 608 may be made of a flexible material, permitting the catheter to be threaded through vasculature or other body cavity of a patient.

In one or more embodiments, multi-electrode assembly 600 is adapted to conform to one or more specific tissues (e.g., cardiac tissue) and also for delivery/removal of the catheter, as described above. Each strut 601, 602, 603, 604, and 605 may independently deflect. The construction of struts 601-605, for example, the length and/or diameter of the struts, and material, may be tailored to achieve desired resiliency, flexibility, foldability, conformability, and stiffness characteristics, along a strut or among the plurality of struts.

While multi-electrode assembly 600 is depicted with ring electrodes 606, embodiments having spot, or other flexible/direct-print electrodes coupled to the struts are readily envisioned, as described above. Ring electrodes 606 can include a single type of electrode or a variety of various electrode types, of diverse sizes. For example, electrodes with smaller surface area may be used exclusively for electrophysiology mapping, while larger surface area electrodes may be used for mapping, tissue ablation, and/or localization. In some cases, the multi-electrode assembly includes one or more slightly enlarged ring electrodes (e.g., for localization of the flexible array in mapping and navigation systems).

Electrodes 606 may be used in various bipole configurations to facilitate measurement of electrical characteristics of tissue in contact with the electrodes. Orthogonally-oriented bipole pair combinations are capable of measuring the unique orientation specific electrical characteristics of the tissue in two orthogonal orientations. A first bipole pair may include a pair of electrodes along the length of struts 601, 602, 603, 604, and 605 facilitating the collection of tissue electrical characteristic data in an orientation substantially parallel with the catheter's longitudinal axis. A second, orthogonal bipole pair may extend laterally across adjacent struts 601 and 602, 602 and 603, 603 and 604, and 604 and 605, facilitating the collection of tissue electrical characteristic data in an orientation substantially transverse to the catheter's longitudinal axis. To facilitate collecting this electrical data, these bipole electrode pairs may be independently addressable by signal processing circuitry. The signal processing circuitry analyzes the received signals from the two sets of bipole pairs to determine orientation independent electrophysiology information of the tissue in contact with the electrodes.

As discussed above, it is possible to drive ablation current between the interelectrode regions of struts 601-605, and optionally between electrodes 606 if desired. The array is configured for bipolar ablation, or, alternatively to drive ablation current in unipolar mode between one or more interelectrode regions and, for example, a patch electrode located on a patient. Unipolar or bipolar ablation therapy technique utilizing interelectrode regions, with or without one or more of electrodes 606 may be used to create specific lesion lines or lesion patterns. For example, struts 601 and 605 can be energized together to form an outer loop and struts 604 and 602 can be energized together to form an inner loop of opposite polarity in one embodiment. Strut 603 can be selectively energized to have the same polarity as the outer loop. Struts 601, 603 and 605 can be energized as cathodes and struts 602 and 604 can be energized as anodes. A specific energization pattern of struts and optionally electrodes mounted thereupon can be implemented to produce a desired lesion surface area (e.g., continuous v. non-continuous lesions, with regularly spaced gaps of untreated tissue), lesion volume, and/or lesion depth.

As with the other electrode arrangements described herein, electrodes in multi-electrode assembly 600 may be selectively activated in unganged or ganged configurations to facilitate electrophysiology, ablation, and/or location determination applications as appropriate.

As shown in FIG. 1H, non-planar arrangements of electrode support members are also provided. For example, multi-electrode assembly 700 represents the distal end of a basket-type catheter. Multi-electrode assembly 700 includes a plurality of electrode support members 702 (hereafter referred to as splines), each of which include a plurality of electrodes 706. Splines 702 may collapse for guidance through an introducer, and expand once the basket extends from the distal end of the introducer. Each of electrodes 706 may be configured for global mapping and/or regional mapping, consistent with various embodiments of the present disclosure.

Splines 702 are coupled to catheter shaft 708 at a proximal end and extend to a distal coupler 714 at a distal end, where the splines terminate. Splines 702 are evenly circumferentially distributed for global mapping as shown, however, in various embodiments of the present disclosure, one or more of the splines may be adjusted to increase or reduce electrode density in part of the array. While the present embodiment presents a basket comprised of eight splines, basket catheters with three or more splines are readily envisioned, with the design depending on an intended clinical application and/or desired electrophysiology mapping granularity. To facilitate expansion/contraction of the basket, a deployment member 704 extends along a longitudinal axis of the basket, which may be pull-wire which extends between a catheter handle and distal coupler 714. Actuation of the pull-wire causes expansion/contraction of the basket. In other embodiments, the deployment member 704 may be a lumen that can be actuated by a manipulator on the catheter handle to expand/contract the basket. In some cases, splines 702 are formed of a shape-memory alloy (e.g., nitinol) which allows the basket to return to a spherical shape after exiting an introducer.

Electrodes 706 are distributed about a length of each spline. While the embodiment presented in FIG. 1H depicts electrodes 706 regularly distributed along the length of each spline, other embodiments may include unevenly distributed electrodes along the splines. For example, only a distal portion of the basket may be in contact with tissue to be treated, a distribution of electrodes 706 may be weighted toward a distal end of basket 700 to facilitate mapping in the tissue. Electrodes 706 can be sized and spaced to ensure that the exposed interelectrode regions of splines 702 are closely spaced and capable of delivering an electric field strength of about 0.1 to 1.0 kV/cm.

Each spline includes a proximal and distal region having outer layer 712 formed of or constructed from an electrically insulating material as described above. A first region of outer layer 712 extends continuously over a distal portion of struts 702 and a second region of outer layer 712 extends distally from shaft 708 to the proximal terminus of electrode 706.

Multi-electrode assembly 700 includes exposed interelectrode regions of splines 702 configured to deliver ablation energy to tissue in contact therewith. Each interelectrode region of splines 702 may be paired with another adjacent interelectrode region to facilitate bipolar tissue ablation, along the same spline or between adjacent splines. Alternatively, or concurrently, interelectrode regions of splines 702 may be paired with a patch electrode (not shown) to operate in a monopolar ablation therapy configuration. The size and spacing of electrodes 706 can be varied to define the area of the exposed interelectrode region, and thereby producing a desired lesion surface area, lesion volume, and/or lesion depth.

Electrodes 706 may be used in various bipole configurations to facilitate measurement of electrical characteristics of tissue in contact with the electrodes. Orthogonally-oriented bipole pair combinations are capable of measuring the unique orientation specific electrical characteristics of the tissue in two orthogonal orientations. A first bipole pair may include a pair of electrodes along a length of spline 702, facilitating the collection of tissue electrical characteristic data in an orientation substantially parallel with the catheter's longitudinal axis. A second, orthogonal bipole pair may extend laterally across adjacent splines 702, facilitating the collection of tissue electrical characteristic data in an orientation substantially transverse to the catheter's longitudinal axis. To facilitate collecting this electrical data, these bipole electrode pairs may be independently addressable by signal processing circuitry. The signal processing circuitry analyzes the received signals from the two sets of bipole pairs to determine orientation independent electrophysiology information of the tissue in contact with the electrodes.

As with the other electrode arrangements described herein, electrodes in multi-electrode assembly 700 may be selectively activated in unganged or ganged configurations to facilitate electrophysiology, ablation, and/or location determination applications as appropriate.

In some embodiments, electrodes 706 can be 0.8 millimeters in diameter with a total surface area of 0.5 mm2. Electrodes 706 on multi-electrode assembly 700 need not be uniform in size and shape. For example, embodiments consistent with the present disclosure may include smaller size electrode(s) (e.g., 0.8 mm in diameter) for electrophysiology mapping, and larger size electrode(s) that may be capable of both electrophysiology mapping, have a large enough impedance to facilitate localization in an impedance or hybrid-based catheter navigation system, or ablation. In some cases, smaller electrophysiology mapping electrodes may be coupled to an tissue-facing surface of the splines for direct contact with tissue, with larger, non-contact navigation electrodes coupled to an internal-facing surface of splines 702. Some of electrodes 706 on multielectrode assembly 700 may be multi-purpose, while other electrodes are single-purpose. For example, some of the electrodes may function as ablation and navigation or electrophysiology mapping electrodes, others may function only as electrophysiology mapping electrodes, and yet other electrodes may function only as navigation electrodes. A specific energization pattern of interelectrode regions and optionally electrodes mounted thereupon can be implemented to produce a desired lesion surface area (e.g., continuous v. non-continuous lesions, with regularly spaced gaps of untreated tissue), lesion volume, and/or lesion depth.

While depicted with equal spacing between all of electrodes 706 both on and between splines 702, the relative spacing between each of the electrodes which form bipole pairs can be varied to accurately capture orientation-specific electrical characteristic data of tissue in contact with the electrodes. In some embodiments, an edge-to-edge spacing for one or more of the bipole pairs of electrodes may be between 2-2.5 mm, and/or center-to-center spacing of the electrodes in a bipole pair may be between 0.5-4 mm.

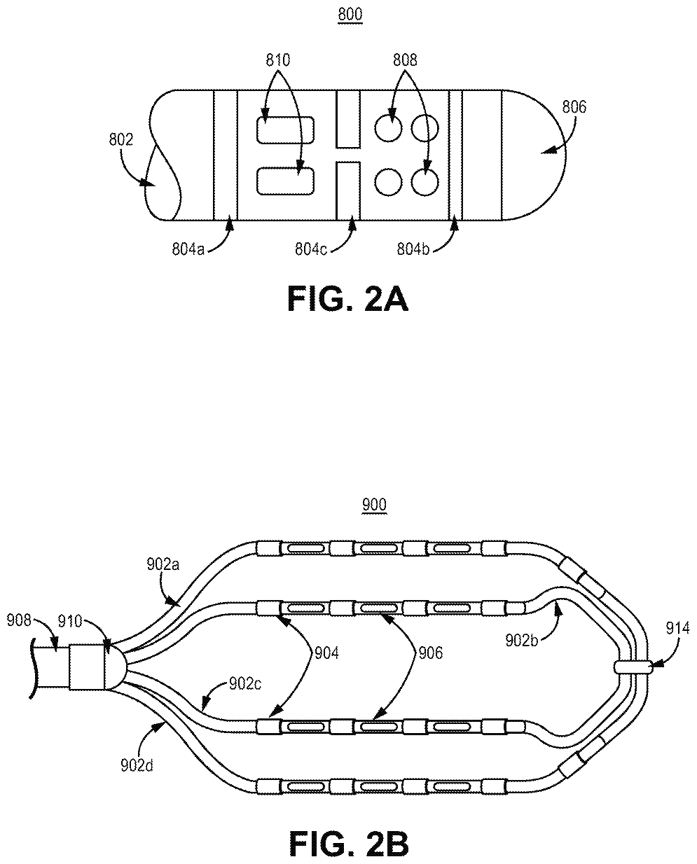

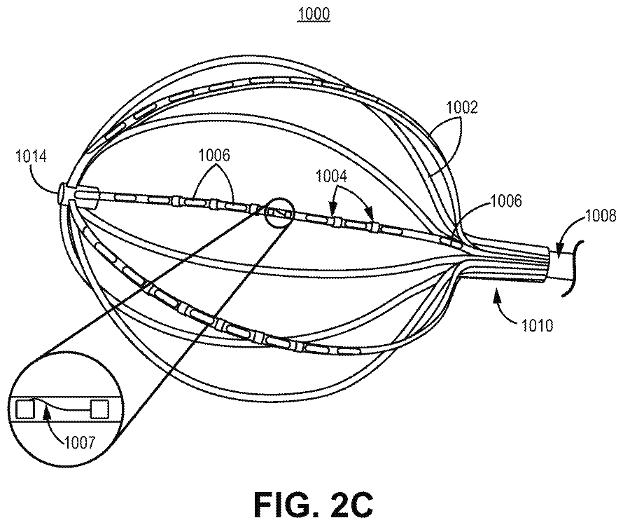

Referring now to FIGS. 2A-2C. embodiments of the present disclosure include a multi-electrode assembly configured to deliver ablation energy and to receive electrophysiological information via electrodes mounted thereupon. In some cases, the ablative energy is delivered solely by electrodes attached to one or more surfaces of the assembly.

For example, FIG. 2A shows a multi-electrode assembly 800 of a linear hybrid electrophysiology mapping/ablation catheter, including electrode support member 802 having mounted (or otherwise affixed) along the longitudinal axis a first plurality of electrodes 804a, 804b, and 804c configured and arranged to deliver ablation energy and a second plurality of electrodes 810 and 808 configured to detect electrophysiological characteristics of tissue. Electrode 804b is positioned proximal to tip electrode 806 at the distal end of assembly 800. As shown, tip electrode 806 is a single unit, however, a plurality of discrete electrode segments are within the scope of this embodiment, as described for multi-electrode assembly 200. Tip electrode 806 can be configured for electrophysiological sensing, measurement, diagnostics and/or for therapy delivery.

Electrodes 804a, 804b, 804c, 806, 810, and/or 808 can be mounted on electrode support member 802. Electrode support member 802 can be coated with a layer of electrically-insulative material. Electrodes 804a-c and 806 can be selected from the durable, non-printed electrodes described above. For example, electrodes 804a and 804b can be ring electrodes, electrode 804c can be a split ring electrode or ring segment electrode, and tip electrode 806 can include a conductive shell. Each of electrodes 804a, 804b, 804c, 806, 810, and/or 808 can include the same or different electrode components mounted in or on the shell as an adjacent electrode. For example, electrodes with smaller surface area may be used exclusively for electrophysiology mapping, while larger surface area electrodes may be used for mapping, tissue ablation, and/or localization. In some cases, the multi-electrode assembly includes one or more slightly enlarged ring electrodes (e.g., for localization of the flexible array in mapping and navigation systems). Split ring electrode 804c does not fully circumscribe the electrode support member on which it is mounted, but is attached to the electrode support member so as to not become detached therefrom upon advancement of the electrode support member through the patient's body (e.g., vasculature). One or more of electrodes 804a and 804b can be segmented and/or all of 804a, 804b, and 804c can be ring electrodes. The number and size of the electrodes can be varied based on the desired therapy. In some cases, electrode support member may include more than or less than four electrodes.

Electrodes 804a, 804b, 804c, and/or 806 may be configured as a bipolar or multipolar electrode assembly for use in bipolar-or multipolar-based electroporation therapy. For example, 804a-c and 806 may be individually electrically coupled to an electroporation generator, such as a power supply via suitable electrical wire or other suitable electrical conductors extending through the catheter shaft and may be configured to be selectively energized (e.g., by a power supply and/or computer system) with opposite polarities to generate a potential and corresponding electric field therebetween, for PFA therapy. One of 804a, 804b, and 804c may be configured to function as a cathode, and another one of the electrodes may be configured to function as an anode. The shape, size, and/or configuration of therapy ablation members may impact various parameters of the applied electroporation therapy. In some cases, the electrodes are configured to perform unipolar ablation.

Multi-electrode assembly 800 may be configured as a monopolar electrode assembly and use a patch electrode (e.g., return electrode (not shown)) as a return or indifferent electrode. For example, the current path may be directed from distal tip electrode 806 to a return electrode, in a monopolar therapy application.

Distal tip electrode 806, or a segment thereof, can be selectively energized independently of electrodes 804a-c. In some embodiments, distal tip electrode 806 is energized with an opposite polarity to one of electrodes 804a, 804b, and 804c, to generate a potential and corresponding electric field therebetween, for PFA therapy. Where distal tip electrode 806 is energized with an opposite polarity to distal electrode 804b, the electric field generated therebetween may be suitable for point PFA therapy, to precisely target tissue adjacent to distal end of multielectrode assembly 800.

At least one area of electrode support member 802 has affixed thereon two pluralities of flexible electrodes 808 and 810. A portion of one or both of the pluralities of flexible electrodes 808 and 810 is configured to detect at least electrophysiological characteristics of the tissue. For example, a portion of flexible electrodes 808 and 810 may also be configured for use with sensors (e.g., position, pacing, strain, temperature, and the like, as described for multi-electrode assembly 300 above). Electrode support member 802 may include flexible thin films compatible with printed circuit manufacturing techniques and/or have such thin films coupled to structural elements of the electrode support member (e.g., nitinol-or polymer-based structural elements). Spot electrodes may be printed onto the electrode support member itself. Printed electrodes may be electrically coupled to signal processing circuitry and/or driver circuitry via traces extending on or within the one or more thin film layers.

Electrodes 808 and 810 can be direct print electrodes. The arrangement and spacing of flexible electrodes 808 and 810 is merely exemplary—other arrangements are within the scope of this embodiment. For example, electrodes 808 and 810 can be positioned to enhance electrophysiological activity measurement in the tissue. By varying the size (e.g., width) and spacing of the flexible and/or direct print electrodes, different diagnostic and/or therapeutic objectives and/or outcomes can be achieved. Inter-electrode spacing can vary along the length of the assembly. The flexible and/or direct print electrodes 808 and 810 can be single sided electrodes or electrodes printed on a flexible, bendable material. In some cases, flexible and/or direct print electrodes are spot electrodes printed over at least a partial surface of electrode support member 802, using a conductive ink. Flexible electrodes can wrap around the entire surface of the electrode support member.

Electrode support member 802 can further include conductive traces (not shown) formed using an ink jet printing process or an additive manufacturing process. In one or more embodiments, printed conductive traces can be used to electrically connect electrodes to conductors, or printed traces could function as conductors of multi-electrode assembly 800. Use of flexible and/or printed electrodes permits a higher density of electrodes, allows for complex and complicated physical patterns of electrodes, and does not reduce array flexibility to the extent of durable, non-printed electrodes. Increasing the number of measurement points for electrophysiological activity is advantageous because it reduces the amount of time required to generate an electrophysiology map using an ablation catheter.

Each of flexible electrodes 808 and 810 can be coupled to and communicatively coupled to signal processing circuitry via electrical traces that extend along interior or exterior layers of a flexible printed circuit board (not shown). Flexible electrodes can be disposed upon an electrically-insulative substrate. Multi-electrode assembly 800 can include any number of flexible electrodes. For example, the plurality of electrodes can include 2, 3, 5, 8, 12, 72, 84 or more flexible and/or direct print electrodes.

As with the other electrode arrangements described herein, electrodes in multi-electrode assembly 800 may be selectively activated in unganged or ganged configurations to facilitate electrophysiology, ablation, and/or location determination applications as appropriate.

Another exemplary embodiment configured to deliver ablation energy and to receive electrophysiological information by electrodes mounted thereupon is shown in FIG. 2B. FIG. 2B shows multi-electrode assembly 900 of a hybrid electrophysiology mapping/ablation catheter, which includes electrode support members 902a, 902b, 902c, and 902d (referred to as “struts” hereafter) coated with an electrically insulating material (e.g., dielectric). Suitable material includes, without limitation, polyethylene terephthalate (PET), parylene, polyimide (e.g., PI-2771 or HD-4004 available from HD Microsystems), polyether block amide (PEBA) and/or an adhesive (e.g., SU8 epoxy available from MicroChem Corp). Where the electrode support member is made of electrically conductive material, the dielectric can electrically insulate electrodes that are mounted thereon from the electrically conductive material. Thus, a particular electrode can independently perform a specialized function (e.g., a mapping function). In some cases, independent electrodes can also be electrically grouped together to perform an additional function, e.g., to behave as a large electrode for ablation/localization.

Multi-electrode assembly 900 includes a planar array of a first plurality of electrodes 904 and a second plurality of electrodes 906 on struts 902a, 902b, 902c, and 902d with the spacing of the electrodes along a length of the strut being the same (or at least known). Other embodiments may include unevenly distributed electrodes along the struts. For example, if only a distal portion of the planar array will be in contact with the tissue to be treated, a distribution of electrodes 904 and 906 may be weighted toward a distal end of assembly 900 to facilitate mapping or therapy delivery in the tissue. The struts extend in a plane that is substantially parallel with a longitudinal axis of catheter shaft 908 and are coupled to one another at distal and proximal ends (e.g., at a distal tip coupler 914 and bushing 910, respectively).

Electrodes 904 are configured to deliver ablation energy. Each of the struts can be precisely, laterally separated from each other to facilitate exact spacing between electrodes 904 on the same strut 902a, 902b, 902c, and/or 902d, and/or between electrodes 904 on adjacent struts 902a-d. The result is a plurality of electrodes forming bipole pairs with known spacings. For example, in some embodiments the center-to-center spacing of a bipole pair may be between 0.5-4 mm, or less than 0.5 millimeters (e.g., 0.1 mm). Alternatively or additionally, the bipole pair spacing can be based on edge-to-edge spacing.

Multi-electrode assembly 900 is configured such that spacing between each respective strut is based on a desired electrode spacing for a given therapy. While the planar array shows 18 electrodes 904, a hybrid electrophysiology mapping/ablation catheter of the present disclosure array may include more or fewer than 18 electrodes. Each strut may have a different number of electrodes than an adjacent strut. In some embodiments, electrodes 904 on adjacent struts 902a-d may perform unipolar or bipolar ablation (e.g., via the use of bipole pairs). This unipolar or bipolar ablation may create specific lines or patterns of lesions.

Electrodes 906 may receive electrical signals from the heart, or other tissue, which can be used for electrophysiological studies/mapping. For example, electrodes 906 capture electrophysiological information, such as the direction and speed of cardiac signals, to enable the creation of high-density electro maps of cardiac tissue to support optimal treatment. The sampled electrical characteristics may be processed to remove catheter orientation-based signal effects. In some embodiments, the electrodes 906 may perform a location or position sensing function related to tissue mapping (e.g., determine location and/or orientation of the catheter).

Planar multi-electrode assembly 900 is coupled to a distal end of a catheter shaft 908 at a bushing 910 (also referred to as a connector). In the present embodiment, each of struts 902a-d extend substantially parallel to the longitudinal axis defined by catheter shaft 908, which may be made of a flexible material, permitting the catheter to be threaded through vasculature or other body cavity of a patient.

In one or more embodiments, multi-electrode assembly 900 is adapted to conform to one or more specific tissues (e.g., cardiac tissue) and also for delivery/removal of the catheter. For example, collapsibility of materials such as nitinol and/or other flexible substrate facilitates insertion of the planar array into a delivery sheath or introducer, whether during delivery of the catheter into the body or removal of the catheter from the body at the end of a procedure. Each strut 902a-d may independently deflect. The construction of struts 902a-d, for example, the length and/or diameter of the struts, and material, may be tailored to achieve desired resiliency, flexibility, foldability, conformability, and stiffness characteristics, along a strut or among the plurality of struts.

Multi-electrode assembly 900 includes a plurality of electrodes 904 as described in one or more embodiments above. For example, electrodes 904 can be selected from ring electrodes, split ring electrodes, and ring segment electrodes. Electrodes 904 may be a single type of electrode or a variety of distinct types of electrodes. Larger surface area electrodes may be used for mapping, tissue ablation, and/or localization. As discussed for multi-electrode assemblies 400-600, it is possible to drive ablation current between electrodes 904 of struts 902a-d, and optionally between electrodes 906 on adjacent struts concurrently, if desired. The array is configured for bipolar ablation, or, alternatively to drive ablation current in unipolar mode between one or more electrodes 904 and, for example, a patch electrode located on a patient. Unipolar or bipolar ablation therapy techniques utilizing electrodes 904, with or without electrodes 906 may be used to create specific lesion lines or lesion patterns.

Multi-electrode assembly 900 further includes a plurality of flexible electrodes 906 as described in one or more embodiments above. Flexible electrodes 906 include spot, or other flexible/direct-print electrodes coupled to the struts. The struts of the planar array may comprise flexible thin films compatible with printed circuit manufacturing techniques and/or have such thin films coupled to structural elements of the strut (e.g., nitinol-based structural elements). Spot electrodes may be printed onto the struts themselves. Printed electrodes may be electrically coupled to signal processing circuitry and/or driver circuitry via traces extending on or within the one or more thin film layers.

Flexible electrodes 906 may be used in various bipole configurations to facilitate measurement of electrical characteristics of tissue in contact with the electrodes. Orthogonally-oriented bipole pair combinations are capable of measuring the unique orientation specific electrical characteristics of the tissue in two orthogonal orientations. A first bipole pair may include a pair of electrodes along the length of struts 902a-d facilitating the collection of tissue electrical characteristic data in an orientation substantially parallel with the catheter's longitudinal axis. A second, orthogonal bipole pair may extend laterally across adjacent struts 902a and 902b, 902b and 902c, 902c and 902d, facilitating the collection of tissue electrical characteristic data in an orientation substantially transverse to the catheter's longitudinal axis. Bipole electrode pairs may be independently addressable by signal processing circuitry. The signal processing circuitry analyzes the received signals from the two sets of bipole pairs to determine orientation independent electrophysiology information of the tissue in contact with the electrodes.

The distal tip includes coupler 914 joining the distal tips formed by the joints connecting struts 902a and 902d and connecting struts 902b and 902c, with or without being electrically connected (as described for multi-electrode assembly 500). Coupler 914 may be constructed from metal or another radiopaque material to permit fluoroscope visualization. The coupler may facilitate (semi-) independent planar movement between the struts while ensuring the struts are secure within the array, e.g., dielectric tubing, maintain the spaced relationship of the array and the electrodes thereon, and permitting the struts to be independently energized For example, struts 902a and 902d can be energized together to form an outer loop and struts 902b and 902c can be energized together to form an inner loop of opposite polarity in one embodiment, or struts 902a and 902c can be energized as cathodes and struts 902b and 902d can be energized as anodes, such that the adjacent struts have opposite polarity. A specific energization pattern of struts and optionally electrodes mounted thereupon can be implemented to produce a desired lesion surface area (e.g., continuous v. non-continuous lesions, with regularly spaced gaps of untreated tissue), volume, and/or depth. Alternatively, one or more of the joints connecting struts 902a and 902d and struts connecting 902b and 902c can electrically connect the respective struts.

As with the other electrode arrangements described herein, electrodes in multi-electrode assembly 900 may be selectively activated in unganged or ganged configurations to facilitate electrophysiology, ablation, and/or location determination applications as appropriate.

Another embodiment of a multi-electrode assembly is configured to deliver ablation energy and to receive electrophysiological information by electrodes mounted thereupon is shown in FIG. 2C. FIG. 2C shows the distal end of a basket-type catheter. Multi-electrode assembly 1000 includes a plurality of electrode support members 1002 (also referred to as splines), each of which include a first plurality of electrodes 1004 and a second plurality of electrodes 1006. Splines 1002 may collapse for guidance through an introducer, and expand once the basket extends from the distal end of the introducer. Each of electrodes 1004 may be configured for delivery of ablation energy and each of electrodes 1006 may be configured for global mapping and/or regional mapping, consistent with various embodiments of the present disclosure.

Splines 1002 are coupled to catheter shaft 1008 at a proximal end and extend to a distal coupler 1010 at a distal end, where the splines terminate. Splines 1002 are depicted as evenly circumferentially distributed for global mapping, however, one or more of splines may be positioned to facilitate more or less electrode density. While the present embodiment presents a basket comprised of eight splines, basket catheters with three or more splines are readily envisioned, with the design depending on an intended clinical application and/or desired electrophysiology mapping granularity. To facilitate expansion/contraction of the basket, a deployment member (not shown) can extend along a longitudinal axis of the basket, which may be pull-wire or a lumen which extends between a catheter handle and distal coupler 1010, as described for multi-electrode assembly 700, causes expansion/contraction of the basket. In some cases, splines 1002 are formed of a shape-memory alloy (e.g., nitinol) which allows the basket to return to a spherical shape after exiting an introducer.

Electrodes 1004 and 1006 are distributed about a length of each spline, evenly, unevenly, or biased towards one end, based on the tissue to be treated. Although electrodes 1004 are positioned between pairs of electrodes 1006, the placement and spacing of electrodes 1004 may vary based on the tissue type, the desired therapy, and the lesion to be created.

Multi-electrode assembly 1000 includes a plurality of electrodes 1004 as described above. For example, electrodes 1004 can be selected from ring electrodes, split ring electrodes, and ring segment electrodes. Electrodes 1004 can be selected from the same type of robust electrode or a variety of distinct types of electrodes. For example, electrodes with smaller surface area may be used exclusively for electrophysiology mapping, while larger surface area electrodes may be used for mapping, tissue ablation, and/or localization. In some cases, the multi-electrode assembly includes one or more slightly enlarged ring electrodes (e.g., for localization of the flexible array in mapping and navigation systems). In some cases, the ablation energy is delivered solely by electrodes 1004. Each electrode 1004 may be paired with another adjacent electrode to facilitate bipolar tissue ablation, along the same spline or between adjacent splines. Alternatively, or concurrently, electrode 1004 may be paired with a patch electrode (not shown) to operate in a monopolar ablation therapy configuration.

Electrodes 1004 on basket 1000 need not be uniform in size and shape. For example, embodiments consistent with the present disclosure may include smaller size electrode(s) (e.g., 0.8 mm in diameter) for electrophysiology mapping, and larger size electrode(s) that may be capable of both electrophysiology mapping, have a large enough impedance to facilitate localization in an impedance or hybrid-based catheter navigation system, or ablation.

In one some cases, smaller electrophysiology mapping electrodes may be coupled to an tissue-facing surface of the splines for direct contact with tissue, with larger, non-contact navigation electrodes coupled to an internal-facing surface of splines 1002. Some of electrodes 1004 on basket 1000 may be multi-purpose, while other electrodes are single-purpose. For example, some of the electrodes may function as ablation and navigation or electrophysiology mapping electrodes, others may function only as electrophysiology mapping electrodes, and yet other electrodes may function only as navigation electrodes. The size and spacing of electrodes 1004 can be varied for flexibility of the array and to define the area for electrodes 1006.

Multi-electrode assembly 1000 further includes a plurality of flexible electrodes 1006 as described for embodiments 300, 800, and others. Flexible electrodes 1006 include spot, or other flexible/direct-print electrodes coupled to at least one surface of the splines. The splines of the basket array may comprise flexible thin films compatible with printed circuit manufacturing techniques and/or have such thin films coupled to structural elements of the strut (e.g., nitinol-based structural elements). Spot electrodes may be printed onto the struts themselves. Printed electrodes may be electrically coupled to signal processing circuitry and/or driver circuitry via traces 1007 in close up view; extending on or within the one or more thin film layers.

Electrodes 1006 may be used in various bipole configurations to facilitate measurement of electrical characteristics of tissue in contact with the electrodes. Orthogonally-oriented bipole pair combinations are capable of measuring the unique orientation specific electrical characteristics of the tissue in two orthogonal orientations. A first bipole pair may include a pair of electrodes along a length of spline 1002, facilitating the collection of tissue electrical characteristic data in an orientation substantially parallel with the catheter's longitudinal axis. A second, orthogonal bipole pair may extend laterally across adjacent splines 1002, facilitating the collection of tissue electrical characteristic data in an orientation substantially transverse to the catheter's longitudinal axis. To facilitate collecting this electrical data, these bipole electrode pairs may be independently addressable by signal processing circuitry. The signal processing circuitry analyzes the received signals from the two sets of bipole pairs to determine orientation independent electrophysiology information of the tissue in contact with the electrodes.

As with the other electrode arrangements described herein, electrodes in multi-electrode assembly 1000 may be selectively activated in unganged or ganged configurations to facilitate electrophysiology, ablation, and/or location determination applications as appropriate.

Multi-electrode assemblies 100, 200, 300, 400, 500, 600, 700, 800, 900, and 1000 are suitable for use in catheters for diagnostic purposes, anatomical mapping and/or ablation therapy (e.g., electroporation therapy). A multi-electrode assembly of the present disclosure, which is capable of both electrophysiology mapping and ablation therapy, may reduce surgery time by eliminating the need to swap out the electrophysiology mapping catheter with an ablation catheter after confirming a treatment strategy. In addition, a multi-electrode assembly of the present disclosure can be used to deliver ablation energy suitable for irreversible electroporation to destroy tissue, in particular, for electroporation-induced primary necrosis therapy. In some cases, where electrodes, exposed interelectrode regions of struts or splines (e.g., tissue ablation members) are closely spaced, electric current can be delivered as a pulsed electric field (i.e., pulsed field ablation (PFA)) in the form of short-duration pulses (e.g., 0.1 to 20 ms duration) capable of delivering an electric field strength of about 0.1 to 1.0 kV/cm.

Advantages of a catheter of the present disclosure include, but are not limited to, robust, yet maneuverable configurations to facilitate high-density mapping of electrophysiological activity in hard-to-reach areas, before and after ablation, without compromising the speed with which maps can be generated, or the efficiency of therapy delivery. The high power ablation and efficient remapping capabilities of the disclosed catheters may improve procedure outcome. For example, embodiments of the present disclosure allow for tissue specific lesion formation within a shortened procedure time.

The preceding examples are intended to illustrate the above invention and should not be construed as to narrow its scope. The scope of this disclosure should be determined by the appended claims and their legal equivalents. Variations and modifications may be made while remaining within the scope of one or more embodiments of the present disclosure. Various features and aspects of the disclosed embodiments can be combined with or substituted for one another in order to form various embodiments.

The terms “proximal” and “distal” are be used throughout the specification with reference to a clinician manipulating one end of an instrument used to treat a patient. Thus, the term “proximal” refers to the portion of the instrument closest to the clinician and the term “distal” refers to the portion located furthest from the clinician. Surgical instruments may be used in many orientations and positions, however, and these terms are not intended to be limiting and absolute.

Patent literature, or other disclosure material, in whole or in part, which is said to be incorporated by reference herein is incorporated herein to the extent that the incorporated materials do not conflict with existing definitions, statements, or other disclosures set forth in herein.

What is claimed is:

1. A multi-electrode assembly of a mapping and ablation catheter, the assembly comprising:
a first plurality of electrodes; and
an electrode support member;
wherein a portion of the electrode support member is adapted to deliver ablation therapy, and wherein at least a portion of the first plurality of electrodes are configured and arranged on the electrode support member to detect electrophysiological characteristics of a tissue in contact therewith.

2. The multi-electrode assembly of claim 1, wherein the first plurality of electrodes is selected from the group consisting of ring electrodes, tip electrodes, tip-segment electrodes, split ring electrodes, and ring-segment electrodes.

3. The multi-electrode assembly of claim 2, wherein the electrode support member further comprises a plurality of spot electrodes, printed electrodes, conductive traces, or a combination thereof.

4. The multi-electrode assembly of claim 3, wherein the electrode support member further comprises a tip electrode configured to deliver ablation energy to the tissue.

5. The multi-electrode assembly of claim 1, wherein the electrode support member is configured as a planar array including two or more struts, each of the struts lying in a common plane, wherein at least a portion of the plurality of electrodes are configured and arranged on the struts to detect electrophysiological characteristics of the tissue in contact with the planar array.

6. The multi-electrode assembly of claim 5, wherein at least one of the first plurality of electrodes includes electrodes formed on an electrically-insulative substrate positioned on a surface of the strut.

7. The multi-electrode assembly of claim 5, wherein the first plurality of electrodes is selected from the group consisting of ring electrodes, split ring electrodes, and ring-segment electrodes.

8. The multi-electrode assembly of claim 7, further comprising a second plurality of electrodes selected from the group consisting of spot electrodes, printed electrodes, and a combination thereof, wherein the second plurality are configured and arranged to detect electrophysiological characteristics of the tissue in contact therewith.

9. The multi-electrode assembly of claim 8, wherein at least one electrode of the first plurality is positioned between two electrodes of the second plurality.

10. The multi-electrode assembly of claim 5, wherein the first plurality of electrodes is selected from the group consisting of spot electrodes, printed electrodes, and a combination thereof.

11. The multi-electrode assembly of claim 5, wherein the first plurality of electrodes are uniformly spaced along the struts.

12. The multi-electrode assembly of claim 11, wherein a surface of the struts comprises exposed metal, wherein the surface is capable of delivering ablation energy between the at least two struts.

13. The multi-electrode assembly of claim 5, wherein the planar array includes at least four struts, and each strut includes at least four electrodes.

14. The multi-electrode assembly of claim 1, wherein the electrode support member is configured as a flexible basket, including a plurality of splines, wherein the first plurality of electrodes are configured and arranged on the splines to detect electrophysiological characteristics of a tissue in contact therewith.

15. The multi-electrode assembly of claim 14, wherein at least one of the electrodes comprises a dielectric layer positioned between the electrode and a spline surface.

16. The multi-electrode assembly of claim 14, wherein the first plurality of electrodes is selected from the group consisting of ring electrodes, split ring electrodes, and ring-segment electrodes.

17. The multi-electrode assembly of claim 16, further comprising a second plurality of electrodes mounted to a surface of the splines, the second plurality selected from the group consisting of spot electrodes, printed electrodes, and a combination thereof, wherein the second plurality are configured and arranged to detect electrophysiological characteristics of the tissue in contact therewith.

18. The multi-electrode assembly of claim 17, wherein at least one electrode of the first plurality is positioned between two electrodes of the second plurality.

19. The multi-electrode assembly of claim 16, wherein the first plurality of electrodes includes at least two sets of electrodes, wherein a first set of electrodes has a larger total surface area than a second set of electrodes.

20. The multi-electrode assembly of claim 14, wherein the first plurality of electrodes is selected from the group consisting of spot electrodes, printed electrodes, and a combination thereof.

21. The multi-electrode assembly of claim 14, wherein a surface of the splines comprises exposed metal, wherein the surface is capable of delivering ablation energy between at least two splines.

22. The multi-electrode assembly of claim 21, wherein the first plurality of electrodes includes at least two sets of electrodes, wherein a first set of electrodes has a larger total surface area than a second set of electrodes.

23. The multi-electrode assembly of claim 22, wherein at least the first set of electrodes is configured to behave as a single large electrode.

24. The multi-electrode assembly of claim 1, wherein at least some electrodes in a group of electrodes including the first plurality of electrodes and the electrode support member are configured to be activated independent from one another in an unganged configuration.

25. The multi-electrode assembly of claim 1, wherein at least some electrodes in a group of electrodes including the first plurality of electrodes and the electrode support member are configured to be activated in unison in a ganged configuration.
